(19) United States
(12) Patent Application Publication
(10) Pub. No.: US 2022/0225920 A1
(43) Pub. Date: Jul. 21, 2022

(54) SYSTEMS AND METHODS FOR PROCESSING BIOLOGICAL SIGNALS

(71) Applicant: Elemind Technologies, Inc., Cambridge, MA (US)

(72) Inventors: Heather Read, Cambridge, MA (US); David Wang, Cambridge, MA (US); Meredith Perry, Cambridge, MA (US); Scott Bressler, Cambridge, MA (US)

(73) Assignee: Elemind Technologies, Inc., Cambridge, MA (US)

(21) Appl. No.: 17/579,194

(22) Filed: Jan. 19, 2022

Related U.S. Application Data
(60) Provisional application No. 63/139,354, filed on Jan. 20, 2021.

(51) Int. Cl. A61B 5/291; A61B 5/00; A61B 5/296; A61B 5/375; A61B 5/304

(52) U.S. Cl. CPC A61B 5/291; A61B 5/0042; A61B 5/7405; A61B 5/375; A61B 5/304; A61B 5/296

(57) ABSTRACT

The present disclosure provides a system for processing biological signals. The system may comprise a sensing module comprising one or more sensors for detecting at least one of a biological parameter of a subject and one or more biological signals of the subject, and an additional sensor for detecting ambient conditions associated with a surrounding environment of the subject. The system may comprise a signal processing module in communication with the sensing module, wherein the processing module is configured to aggregate and process data obtained using the one or more sensors to compute one or more markers for the subject. The system may comprise an output device optimization module in communication with the signal processing module and one or more output devices, wherein the output device optimization module is configured to control the output devices using the one or more computed markers and data obtained using the additional sensor.

### CROSS-REFERENCE TO RELATED APPLICATIONS

This application claims the benefit of U.S. Provisional Application No. 63/139,354 filed Jan. 20, 2021 (Attorney Docket No. 40415.04002), the entire contents of which is hereby incorporated by reference.

### BACKGROUND

Brain signals and brain waves can be detected and monitored to determine one or more states of a subject. Ergodic or oscillatory brain signals and brain waves can correspond to one or more electrical signals generated by and/or transmitted between one or more neurons in a subject's brain. The brain signals and brain waves can comprise different types of signals and/or waves corresponding to different brain states of a subject.

### SUMMARY

Recognized herein are various limitations with existing brain computer interface and neurofeedback technologies that are commercially available, which rely on inefficient, inaccurate and non-individualized Artificial Intelligence (AI) and Machine Learning (ML) algorithms that cannot perform instantaneous updating and optimization of operations based on dynamic biological signals (e.g., brain signals and/or other bodily signals) that are highly variable across individuals and over time as observed in natural human physiology. Commercially available technologies typically filter and process raw brain signals utilizing frequency ranges observed across an entire range of sexes and genders in the human population, and are not tailored to individual users or subjects. Furthermore, existing technologies generally control device output in a pre-determined or otherwise restricted and non-dynamic manner over an extended and non-specific time period. Moreover, such technologies are not optimized based on ongoing and prior brain activity of an individual subject. The present disclosure provides systems and methods that can be implemented to overcome these disadvantages inherent in commercially available technologies.

The present disclosure generally relates to the fields of brain computer interface, neurofeedback, and biological device control. More specifically, the present disclosure relates to systems and methods for tracking and computing biological signals (e.g., instantaneous endogenous brain signals and/or other bodily signals) and metrics on multiple time scales using one or more algorithms. Such algorithms can be implemented to control outputs from devices including but not limited to audio speakers, alarms, computers, video and television monitors, smart phones, etc. Device output may be monitored and adjusted in order to control a subject's environment (including exposure to or consumption of various media or sources of digital entertainment) and/or to modulate a subject's physiological, perceptual, cognitive, and/or behavioral states. The systems and methods of the present disclosure can be utilized to control and optimize closed-loop device output in a manner that is precisely tuned to an individual subject's own neurophysiology. In some embodiments, the systems and methods of the present disclosure can also be utilized to tailor and/or tune device outputs specifically for a particular subject to maximally drive a change in subsequent brainwave characteristics, including brain wave oscillations and amplitudes.

In an aspect, the present disclosure provides automated systems and methods for tracking and classifying instantaneous brain states to control one or more output devices. Device outputs can serve as a closed-loop modulator of neurophysiological, perceptual, cognitive and behavioral states and can provide instantaneous physiologically-driven sensory or other stimulus feedback. Device outputs can also be designed to optimize closed-loop neuromodulation based on instantaneous brain state markers or biomarkers. As used herein, closed-loop may refer to the use or implementation of a closed-loop control system with one or more feedback loops to modulate device outputs and automatically regulate process variables to a desired state or set point. In some cases, the closed-loop control system can comprise a proportional-integral-derivative controller (PID controller). As used herein, a marker may refer to any type of biological marker, identifying feature, or measurable property that indicates a biological, neurological, physiological, perceptual, cognitive, or behavioral state or condition.

In some embodiments, the systems and methods of the present disclosure can be implemented to record and analyze brain electrophysiological signals with computationally efficient algorithms implemented on remote tabletop and/or wearable devices to control outputs from secondary devices (e.g., audio speakers, lights, thermostats, secondary computers, television display monitors, transcranial electrical stimulation devices, etc). Oscillatory brain signals can be recorded using surgically implanted electrodes (which can penetrate one or more membranes surrounding a subject's brain), surface electrode arrays, and/or one or more encephalogram (EEG) electrodes, which may include, for example, external scalp EEG electrodes, or any other type of electrode that can be attached to or placed in contact with a portion of a subject's body (e.g., an ear or a forehead of the subject). The electrodes can stimulate brain tissue (cortex or deeper), or record neural activity, or both. The electrodes may be used alone or together with one or more external recording electrodes. For example, the one or more external recording electrodes may record neural activity that has been affected by stimulation from one or more implantable electrodes. In some cases, a computing device can be used to record brain signals and optionally receive or capture additional sensor data (e.g., data obtained during actigraphy, or data obtained using a thermometer, an oximeter, one or more light sensors, or any other type of sensor). In some cases, the additional sensor data may be captured using one or more optical sensors, temperature sensors, radiation sensors, proximity sensors, pressure sensors, position sensors, photoelectric sensors, vision or imaging sensors, particle sensors, motion sensors, humidity sensors, chemical sensors, force sensors, flow sensors, electrical sensors, or contact sensors. In some cases, the computing device can implement one or more embedded operations to allow real-time tracking and computation of biomarkers used to control a peripheral device output in a closed-loop manner. The embedded operations can be performed in real-time based on instantaneous metrics received and/or processed in real time, and can be optimized iteratively based on newly acquired data or metrics received in real-time. In some cases, such embedded operations can be optimized to modulate and facilitate one or more closed-loop systems based on a plurality of behavioral and physiological states including but not limited to: attention, alertness, relaxation, perceptual ambiance or sleep. Additionally, the one or more embedded operations can be optimized to improve a subject's health condition or performance based on the subject's sex, age, and/or other appropriate electrophysiological sensor data obtained for a normative population. In some embodiments, the one or more embedded operations can be optimized for an individual user or subject based on characteristic individual oscillatory brainwaves and biomarkers, using systems and methods that can optimize performance iteratively during a single use and/or after post-hoc data accumulation with repetitive use.

In some embodiments, the systems and methods of the present disclosure can be used to monitor one or more changes in a subject's brain state according to a gradient descent, a multi-state, and/or a two-state classifier model. The systems and methods of the present disclosure can also be implemented to compute, tailor, and modify one or more brain state device control parameters in real-time based on several factors, including but not limited to: a subject's endogenous center or peak brainwave oscillation frequency, a variance associated with the subject's brainwave oscillations, one or more ratios of specific brainwaves, one or more co-variances across brainwaves, sensor data, and/or a detected presence of extracted biomarkers following a removal and/or a rejection of artifacts and filters. In some embodiments, the systems and methods of the present disclosure can be implemented to tailor and/or modify brain state device control parameters in real-time based on a phase of a subject's brainwave (e.g., an endogenous brainwave that exhibits the largest positive or negative amplitude shift in response to an external or internal stimulus). The systems and methods of the present disclosure may be implemented for many different use cases, including but not limited to therapeutic use cases, treatment, training, and/or entertainment.

In one aspect, the present disclosure provides a system for controlling output devices, comprising: (a) a sensing module comprising (i) one or more sensors for detecting at least one of a biological parameter of a subject and one or more biological signals of the subject, and (ii) an additional sensor for detecting one or more ambient conditions associated with a surrounding environment of the subject, wherein at least one sensor of the sensing module is configured to contact a portion of the subject's body; (b) a signal processing module in communication with the sensing module, wherein the processing module is configured to aggregate and process data obtained using the one or more sensors to compute one or more biomarkers for the subject; and (c) an output device optimization module in communication with the signal processing module and one or more output devices, wherein the output device optimization module is configured to determine an optimal output for the one or more output devices and control an operation of the one or more output devices to provide the optimal output based on (i) the one or more computed biomarkers and (ii) data obtained using the additional sensor.

In some embodiments, the one or more sensors comprise a sensor for detecting the one or more biological signals of the subject, wherein the sensor comprises a surgically implanted electrode, a surface electrode, or an encephalogram (EEG) electrode.

In some embodiments, the one or more biological signals comprise an electroencephalogram (EEG) signal, an electromyogram (EMG) signal, an electrocorticogram (ECoG) signal, or a field potential within a cerebral cortex region of the subject's brain.

In some embodiments, the one or more sensors comprise a sensor for detecting the biological parameter of the subject.

In some embodiments, the additional sensor comprises a sensor configured to detect one or more environmental conditions of the surrounding environment.

In some embodiments, the biological parameter comprises a physical or physiological condition, state, or property of the subject.

In some embodiments, the one or more ambient conditions correspond to a temperature of the surrounding environment, an amount or volume of sound or noise in the surrounding environment, or a lighting condition of the surrounding environment, wherein the lighting condition comprises an amount, an intensity, a directionality, a color, or a temperature of light in the surrounding environment.

In some embodiments, the one or more biomarkers comprise a center frequency of the one or more biological signals.

In some embodiments, the center frequency is computed by applying a transform to the one or more biological signals.

In some embodiments, the center frequency is computed using a 1/f detrended absolute power spectrum by locating a peak or maximum power within a targeted frequency range of the one or more biological signals.

In some embodiments, the one or more biomarkers comprise a ratio between two or more brainwave oscillation frequency bands.

In some embodiments, the one or more biomarkers comprise a measurement of a coherence between two or more brainwave oscillations.

In some embodiments, the one or more biomarkers comprise a measurement of a phase shift or a phase difference between two or more brainwave oscillations.

In some embodiments, the one or more biomarkers comprise a variance or a co-variance associated with one or more brainwave oscillations.

In some embodiments, the output device optimization module is configured to operate or control the one or more output devices in a graded proportional manner.

In some embodiments, the output device optimization module is configured to operate or control the one or more output devices in a switch fashion.

In some embodiments, the output device optimization module is configured to implement an optimization framework for active suppression or amplification of neural oscillations over one or more time-scales using closed-loop stimulation.

In some embodiments, the output device optimization module is configured to control the one or more output devices based on one or more threshold values associated with the one or more biomarkers.

In some embodiments, the one or more threshold values are updated based on an additional set of biological signals or biological parameters obtained for the subject.

In some embodiments, the sensing module comprises a wearable headband.

In some embodiments, the one or more output devices are configured to provide a stimulation to the subject to induce a desired state.

In some embodiments, the desired state corresponds to a state of wakefulness.

In some embodiments, the desired state corresponds to a sleep state.

In some embodiments, the desired state corresponds to a state of attention or a state of alertness.

In some embodiments, the desired state corresponds to a state of relaxation.

In some embodiments, the one or more output devices are configured to provide a stimulation that is phase-locked with a detected instantaneous phase or instantaneous amplitude of the one or more biological signals.

In some embodiments, the one or more biomarkers are updated based on an additional set of biological signals or biological parameters obtained for the subject.

In some embodiments, the one or more output devices are configured to provide a stimulation to the subject to modify a current state of the subject.

In some embodiments, the stimulation comprises auditory, visual, electrical, magnetic, vibrotactile, or haptic stimuli.

In another aspect, the present disclosures provides a method for controlling one or more output devices, comprising: (a) using (i) one or more sensors to detect at least one of a biological parameter of a subject and one or more biological signals of the subject and (ii) an additional sensor to detect one or more ambient conditions associated with a surrounding environment of the subject, wherein at least one of the one or more sensors is placed in contact with a portion of the subject's body; (b) processing the data obtained using the one or more sensors to compute one or more biomarkers for the subject; and (c) controlling an operation of the one or more output devices based on the one or more computed biomarkers and data obtained using the additional sensor.

In some embodiments, the one or more sensors comprise a sensor for detecting the one or more biological signals of the subject, wherein the sensor comprises a surgically implanted electrode, a surface electrode, or an encephalogram (EEG) electrode.

In some embodiments, the one or more biological signals comprise an electroencephalogram (EEG) signal, an electromyogram (EMG) signal, an electrocorticogram (ECoG) signal, or a field potential within a cerebral cortex region of the subject's brain.

In some embodiments, the one or more sensors comprise a sensor for detecting the biological parameter of the subject, wherein the sensor comprises a thermometer, an oximeter, an accelerometer, or a heartbeat sensor.

In some embodiments, the additional sensor comprises an ambient sound sensor, an ambient light sensor, or an ambient temperature sensor.

In some embodiments, the biological parameter comprises a temperature, a pulse, or a heart rate of the subject.

In some embodiments, the one or more ambient conditions correspond to a temperature of the surrounding environment, an amount or volume of sound or noise in the surrounding environment, or a lighting condition of the surrounding environment, wherein the lighting condition comprises an amount, an intensity, a directionality, a color, or a temperature of light in the surrounding environment.

In some embodiments, the one or more biomarkers comprise a center frequency of the one or more biological signals.

In some embodiments, the one or more biomarkers comprise a ratio between two or more brainwave oscillation frequency bands, a measurement of a coherence between two or more brainwave oscillations, a measurement of a phase shift or a phase difference between two or more brainwave oscillations, or a variance or a co-variance associated with one or more brainwave oscillations.

In some embodiments, the method further comprises controlling the one or more output devices in a graded proportional manner.

In some embodiments, the method further comprises controlling the one or more output devices in a switch fashion.

In another aspect, the present disclosure provides a system for modulating brain states, comprising: (a) a sensing module comprising (i) one or more sensors for detecting at least one of a biological parameter of a subject and one or more biological signals of the subject, and (ii) an additional sensor for detecting one or more ambient conditions associated with a surrounding environment of the subject, wherein at least one sensor of the sensing module is configured to contact a portion of the subject's body; (b) a signal processing module in communication with the sensing module, wherein the processing module is configured to aggregate and process data obtained using the one or more sensors to compute one or more biomarkers for the subject; and (c) an output device optimization module in communication with the signal processing module and one or more output devices, wherein the output device optimization module is configured to determine an optimal output for the one or more output devices and control an operation of the one or more output devices to provide the optimal output based on (i) the one or more computed biomarkers and (ii) data obtained using the additional sensor, wherein the one or more output devices are configured to provide the subject with a stimulation to change a current state of the subject or to induce a desired state in the subject.

In some embodiments, the one or more sensors comprise a sensor for detecting the one or more biological signals of the subject, wherein the sensor comprises a surgically implanted electrode, a surface electrode, or an external scalp encephalogram (EEG) electrode.

In some embodiments, the one or more biological signals comprise an electroencephalogram (EEG) signal, an electromyogram (EMG) signal, an electrocorticogram (ECoG) signal, or a field potential within a cerebral cortex region of the subject's brain.

In some embodiments, the one or more sensors comprise a sensor for detecting the biological parameter of the subject, wherein the sensor comprises a thermometer, an oximeter, an accelerometer, or a heartbeat sensor.

In some embodiments, the additional sensor comprises an ambient sound sensor, an ambient light sensor, or an ambient temperature sensor.

In some embodiments, the biological parameter comprises a temperature, a pulse, or a heart rate of the subject.

In some embodiments, the one or more ambient conditions correspond to a temperature of the surrounding environment, an amount or volume of sound or noise in the surrounding environment, or a lighting condition of the surrounding environment, wherein the lighting condition comprises an amount, an intensity, a directionality, a color, or a temperature of light in the surrounding environment.

In some embodiments, the one or more biomarkers comprise a center frequency of the one or more biological signals.

In some embodiments, the center frequency is computed by applying a Fourier transform or a Hilbert transform to the one or more biological signals.

In some embodiments, the one or more biomarkers comprise a ratio between two or more brainwave oscillation frequency bands, a measurement of a coherence between two or more brainwave oscillations, a measurement of a phase shift or a phase difference between two or more brainwave oscillations, or a variance or a co-variance associated with one or more brainwave oscillations.

In some embodiments, the output device optimization module is configured to operate the one or more output devices in a graded proportional manner.

In some embodiments, the output device optimization module is configured to operate the one or more output devices in a switch fashion.

In some embodiments, the output device optimization module is configured to control the one or more output devices based on one or more threshold values associated with the one or more biomarkers.

In some embodiments, the one or more threshold values are updated based on an additional set of biological signals obtained for the subject.

In some embodiments, the sensing module comprises a wearable headband.

In some embodiments, the current state or the desired state corresponds to a state of wakefulness, a sleep state, a state of attention or a state of alertness, or a state of relaxation.

In some embodiments, the stimulation comprises auditory, visual, electrical, magnetic, vibrotactile, or haptic stimuli.

In another aspect, the present disclosure provides a method for modulating brain states, comprising: (a) using (i) one or more sensors to detect at least one of a biological parameter of a subject and one or more biological signals of the subject and (ii) an additional sensor to detect one or more ambient conditions associated with a surrounding environment of the subject, wherein at least one of the one or more sensors is placed in contact with a portion of the subject's body; (b) processing the data obtained using the one or more sensors to compute one or more biomarkers for the subject; and (c) controlling an operation of one or more output devices, based on the one or more computed biomarkers and the data obtained using the additional sensor, to provide a stimulation to the subject to change a current state of the subject or to induce a desired state in the subject.

In some embodiments, the current state or the desired state corresponds to a physiological, perceptual, cognitive, or behavioral state of the subject.

In some embodiments, the method further comprises using the one or more computed biomarkers to track or identify one or more abnormal responses to the stimulation.

In some embodiments, the one or more sensors comprise a sensor for detecting the one or more biological signals of the subject, wherein the sensor comprises a surgically implanted electrode, a surface electrode, or an encephalogram (EEG) electrode.

In some embodiments, the one or more biological signals comprise an electroencephalogram (EEG) signal, an electromyogram (EMG) signal, an electrocorticogram (ECoG) signal, or a field potential within a cerebral cortex region of the subject's brain.

In some embodiments, the one or more sensors comprise a sensor for detecting the biological parameter of the subject, wherein the sensor comprises a thermometer, an oximeter, an accelerometer, or a heartbeat sensor.

In some embodiments, the additional sensor comprises an ambient sound sensor, an ambient light sensor, or an ambient temperature sensor.

In some embodiments, the biological parameter comprises a temperature, a pulse, or a heart rate of the subject.

In some embodiments, the one or more ambient conditions correspond to a temperature of the surrounding environment, an amount or volume of sound or noise in the surrounding environment, or a lighting condition of the surrounding environment, wherein the lighting condition comprises an amount, an intensity, a directionality, a color, or a temperature of light in the surrounding environment.

In some embodiments, the one or more biomarkers comprise a center frequency of the one or more biological signals.

In some embodiments, the one or more biomarkers comprise a ratio between two or more brainwave oscillation frequency bands, a measurement of a coherence between two or more brainwave oscillations, a measurement of a phase shift or a phase difference between two or more brainwave oscillations, or a variance or a co-variance associated with one or more brainwave oscillations.

In some embodiments, the method further comprises controlling the one or more output devices in a graded proportional manner.

In some embodiments, the method further comprises controlling the one or more output devices in a switch fashion.

In some embodiments, the stimulation comprises auditory, visual, electrical, magnetic, vibrotactile, or haptic stimuli.

In another aspect, the present disclosure provides a system for controlling output devices, comprising: (a) a sensing module comprising (i) a first sensor for detecting one or more biological signals of a subject, (ii) a second sensor for detecting a biological parameter of the subject, and (iii) a third sensor for detecting one or more ambient conditions associated with a surrounding environment of the subject, wherein at least one sensor of the sensing module is configured to contact a portion of the subject's body; (b) a signal processing module in communication with the sensing module, wherein the processing module is configured to aggregate and process data obtained using the first sensor and the second sensor to compute one or more biomarkers for the subject; and (c) an output device optimization module in communication with the signal processing module and one or more output devices, wherein the output device optimization module is configured to determine an optimal output for the one or more output devices and control an operation of the one or more output devices to provide the optimal output based on (i) the one or more computed biomarkers and (ii) data obtained using the third sensor.

In another aspect, the present disclosure provides a method for controlling one or more output devices, comprising: (a) using (i) a first sensor to detect one or more biological signals of a subject, (ii) a second sensor to detect a biological parameter of the subject, and (iii) a third sensor to detect one or more ambient conditions associated with a surrounding environment of the subject, wherein at least one of the first sensor, the second sensor, and the third sensor is placed in contact with a portion of the subject's body; (b) aggregating and processing the data obtained using the first sensor and the second sensor to compute one or more biomarkers for the subject; and (c) controlling an operation of the one or more output devices based on the one or more computed biomarkers and data obtained using the third sensor.

In another aspect, the present disclosure provides a system for modulating brain states, comprising: (a) a sensing module comprising (i) a first sensor for detecting one or more biological signals of a subject, (ii) a second sensor for detecting a biological parameter of the subject, and (iii) a third sensor for detecting one or more ambient conditions associated with a surrounding environment of the subject, wherein at least one sensor of the sensing module is configured to contact a portion of the subject's body; (b) a signal processing module in communication with the sensing module, wherein the processing module is configured to aggregate and process data obtained using the first sensor and the second sensor to compute one or more biomarkers for the subject; and (c) an output device optimization module in communication with the signal processing module and one or more output devices, wherein the output device optimization module is configured to determine an optimal output for the one or more output devices and control an operation of the one or more output devices to provide the optimal output based on (i) the one or more computed biomarkers and (ii) data obtained using the third sensor, wherein the one or more output devices are configured to provide the subject with a stimulation to change a current state of the subject or to induce a desired state in the subject.

In another aspect, the present disclosure provides a method for modulating brain states, comprising: (a) using (i) a first sensor to detect one or more biological signals of a subject, (ii) a second sensor to detect a biological parameter of the subject, and (iii) a third sensor to detect one or more ambient conditions associated with a surrounding environment of the subject, wherein at least one of the first sensor, the second sensor, and the third sensor is placed in contact with a portion of the subject's body; (b) aggregating and processing the data obtained using the first sensor and the second sensor to compute one or more biomarkers for the subject; and (c) controlling an operation of one or more output devices, based on the one or more computed biomarkers and data obtained using the third sensor, to provide a stimulation to the subject to change a current state of the subject or to induce a desired state in the subject.

In another aspect, the present disclosure provides a system for modulating brain states, comprising: (a) a sensing module comprising one or more sensors configured to detect one or more biological signals of a subject, wherein at least one of the one or more sensors is placed in contact with a portion of the subject's body; (b) a signal processing module in communication with the sensing module, wherein the signal processing module is configured to compute one or more biomarkers based on the one or more biological signals; and (c) an output device optimization module in communication with the signal processing module and one or more output devices configured to provide a stimulation to the subject, wherein the output device optimization module is configured to: (i) determine an optimal stimulation based on the one or more biomarkers, (ii) control an operation of the one or more output devices to provide the optimal stimulation to the subject, (iii) iteratively update the optimal stimulation based on a detection of one or more instantaneous changes to the one or more biomarkers, and (iv) modify the operation of the one or more output devices in real time to provide the updated optimal stimulation to the subject to induce a desired state in the subject.

In some embodiments, the optimal stimulation comprises an auditory, visual, electrical, magnetic, vibrotactile, or haptic stimuli.

In some embodiments, the one or more biological signals comprise an electroencephalogram (EEG) signal, an electromyogram (EMG) signal, an electrocorticogram (ECoG) signal, or a field potential within a cerebral cortex region of the subject's brain.

In some embodiments, the one or more biomarkers comprise a center frequency of the one or more biological signals.

In some embodiments, the one or more biomarkers comprise a ratio between two or more brainwave oscillation frequency bands.

In some embodiments, the one or more biomarkers comprise a measurement of a coherence between two or more brainwave oscillations.

In some embodiments, the one or more biomarkers comprise a measurement of a phase shift or a phase difference between two or more brainwave oscillations.

In some embodiments, the one or more biomarkers comprise a variance or a co-variance associated with one or more brainwave oscillations.

In another aspect, the present disclosure provides a method for modulating brain states, comprising: (a) using one or more sensors to detect one or more biological signals of a subject; (b) computing one or more biomarkers based on the one or more biological signals; (c) determining an optimal stimulation based on the one or more biomarkers; (d) controlling an operation of one or more output devices to provide the optimal stimulation to the subject; (e) iteratively updating the optimal stimulation based on a detection of one or more instantaneous changes to the one or more biomarkers; and (f) modifying the operation of the one or more output devices in real time to provide the updated optimal stimulation to the subject to induce a desired state in the subject.

In some embodiments, the optimal stimulation comprises an auditory, visual, electrical, magnetic, vibrotactile, or haptic stimuli.

In some embodiments, the one or more biological signals comprise an electroencephalogram (EEG) signal, an electromyogram (EMG) signal, an electrocorticogram (ECoG) signal, or a field potential within a cerebral cortex region of the subject's brain.

In some embodiments, the one or more biomarkers comprise a center frequency of the one or more biological signals.

In some embodiments, the one or more biomarkers comprise a ratio between two or more brainwave oscillation frequency bands.

In some embodiments, the one or more biomarkers comprise a measurement of a coherence between two or more brainwave oscillations.

In some embodiments, the one or more biomarkers comprise a measurement of a phase shift or a phase difference between two or more brainwave oscillations.

In some embodiments, the one or more biomarkers comprise a variance or a co-variance associated with one or more brainwave oscillations.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a software architecture for recording EEG and computing biomarkers to control audio speaker device output in a real-time closed-loop manner, in accordance with some embodiments.

FIG. 2 schematically illustrates a process flow diagram for EEG signal biomarker computation to control audio speaker device output, in accordance with some embodiments.

FIG. 3 schematically illustrates a system for instantaneously tracking EEG brain signal from a single electrode in order to compute real-time biomarkers of alertness versus sleepiness brain-states, in accordance with some embodiments.

FIG. 4 schematically illustrates a system for instantaneously tracking EEG brain biomarkers at separate Frontal (Fpz) and Occipital (O2) channel locations in order to control audible changes in speaker output volume with volitional change in focused alertness, in accordance with some embodiments.

FIG. 5 schematically illustrates a system for instantaneously tracking EEG signals and computing a low frequency (LF) normalized alpha envelope biomarker to set a threshold level for delivery of closed-loop audio output phase-locked to the peak phase of alpha brainwave oscillations, in accordance with some embodiments.

FIG. 6 schematically illustrates a computer system that is programmed or otherwise configured to implement methods for optimizing device outputs based on biological signals.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 1 millisecond (ms), 5 ms, 0.01 seconds, 0.05 seconds, 0.1 seconds, 0.5 seconds, 1 second, 0.1 minute, 0.5 minutes, 1 minute, or more. In some cases, a real time event may be performed almost immediately or within a short enough time span, such as within at most 1 second, 0.5 seconds, 0.1 seconds, 0.05 seconds, 0.01 seconds, 5 ms, 1 ms, or less.

### System

In an aspect, the present disclosure provides a system for modifying, fine tuning, and implementing control parameters for output devices in real time to maximally drive a change in a subject's brain activity to achieve a desired brain state or behavioral state. The desired brain state or behavioral state may be determined or set based on a time of day, a state or condition of the subject's surrounding environment, and/or the subject's actions, habits, tendencies, or behaviors. In some embodiments, the system can be used to continually compute and monitor one or more unique brain signatures across varying time windows to achieve a desired brain state. In some embodiments, the system can be used to adjust one or more output device control parameters based on a user's unique and dynamically changing brain activity. In some embodiments, the system can be used to (i) derive optimal control parameters for stimulation based on a continuous, real-time computation of key individual metrics (e.g., biomarkers) and (ii) tailor the optimal stimulation parameters to a subject's unique and dynamically changing brain activity to maximally drive neuromodulation. In some embodiments, the system can be configured to (i) compute a subject's brain signature and (ii) use the subject's brain signature to implement device parameter optimization. In some cases, the system can be configured to compute a stimulation phase and amplitude that maximizes the suppression or amplification of neural oscillations in a particular subject.

In some embodiments, the control parameters for the output devices can be modified, fine-tuned based on one or more biological signals of the subject. The subject can be a human or an animal (e.g., a dog, a cat, a rodent, or any other non-human living being). The subject can be in or near an environment in which one or more output devices are located, or where an output of the one or more output devices can be perceived by the subject. In some cases, the environment can be an indoor environment. In other cases, the environment can be an outdoor environment.

### Sensing Module

The system can comprise a sensing module. The sensing module can comprise one or more electrodes or electrode arrays. The electrodes or electrode arrays can include, for example, surgically implanted electrodes (which can penetrate one or more membranes surrounding a subject's brain), surface electrode arrays, and/or one or more external scalp encephalogram (EEG) electrodes. The electrodes or electrode arrays can be used to obtain one or more biological signals of the subject.

In some embodiments, the sensing module can comprise a plurality of sensors. The plurality of sensors can be used to obtain additional sensor data pertaining to a physiological or physical condition of the subject. The plurality of sensors can comprise, for example, a heartbeat sensors, a thermometer, an oximeter, and/or one or more light sensors. In some cases, the plurality of sensors can comprise one or more optical sensors, temperature sensors, radiation sensors, proximity sensors, pressure sensors, position sensors, photoelectric sensors, vision or imaging sensors, particle sensors, motion sensors, humidity sensors, chemical sensors, force sensors, flow sensors, electrical sensors, or contact sensors.

The sensing module can be configured to detect, measure, record, quantify, and/or read one or more biological signals of a subject. The one or more biological signals can comprise, for example, brain waves or brain signals. The one or more biological signals can comprise an electrical signal and/or an oscillatory signal. The one or more biological signals can be represented as one or more EEG waves or waveforms (also referred to herein as brain waves or brain signals). The one or more biological signals can include an electroencephalogram (EEG) signal, an electromyogram (EMG) signal, an electrocorticogram (ECoG) signal, field potentials within a motor cortex or other regions of the brain, or combinations thereof. In some cases, the one or more biological signals may comprise electrical signal produced by neural tissue, or a motion such as a muscle tremor. The one or more biological signals can correspond to a particular mental state of the subject. For example, in a first mental state, the subject may exhibit a first set of biological signals with a first set of characteristics, whereas in a second mental state, the subject may exhibit a second set of biological signals with a second set of characteristics. The characteristics associated with the biological signals may comprise, for example, a wavelength, a frequency, an amplitude, a phase, a center frequency, a phase difference, a variance, a co-variance, or any other physical property associated with the one or more biological signals.

The sensing module may comprise one or more attachment devices for securing or coupling the sensing module to a portion of a subject's body. In some cases, the attachment device can comprise, for example, an adjustable strap. The attachment device can be configured to releasably couple the sensing module (or one or more components of the sensing module) to the subject's body to enable the sensing module to read a subject's biological signals.

As described above, the sensing module can comprise a plurality of sensors. In some embodiments, the plurality of sensors (or a subset thereof) can be integrated onto a structural component of the sensing module. In other embodiments, the plurality of sensors (or a subset thereof) can be located remote from the sensing module (e.g., on a portion of the subject's body).

In some cases, the plurality of sensors can be operatively coupled to a processing unit onboard the sensing module (e.g., via a wired or wireless connection, network, or communication protocol). The processing unit can be used to aggregate and/or preprocess the one or more biological signals obtained using the plurality of sensors.

In some embodiments, the sensing module and/or the processing unit of the sensing module can be placed in wireless or wired communication with a processing module as described elsewhere herein. The sensing module can be configured to transmit the one or more biological signals to the processing module. The processing module can comprise a signal processing module that is configured to process the one or more biological signals received from the sensing module to compute one or more biomarkers associated with the biological signals. In some cases, the signal processing module can be configured to process the one or more biological signals received from the sensing module to compute one or more properties or characteristics associated with the one or more biological signals.

In some embodiments, the sensing module can comprise a plurality of submodules. The plurality of submodules can comprise a first submodule configured to measure and/or detect biological signals of the subject. The first submodule can be further configured to measure and/or detect a physical or physiological condition of the subject. The plurality of submodules can further comprise a second submodule configured to detect and/or measure one or more ambient conditions of a surrounding environment in which the subject is located. The one or more ambient conditions can comprise, for example, ambient temperatures, ambient lighting conditions, and/or ambient sound levels.

In some cases, the sensing module can comprise one or more ambient sensors. The one or more ambient sensors may comprise, for example, ambient light sensors, ambient temperature sensors, and/or ambient sound sensors. The ambient sensors can be configured to obtain ambient sensor data corresponding to one or more ambient conditions associated with the surrounding environment in which a subject is located. In some embodiments, the ambient sensor data be used to control an operation of one or more output devices. In other embodiments, the ambient sensor data can be used to fine tune the closed loop control of the one or more output devices.

In some cases, ambient sensor data can be used post-session to understand why a physiological behavior or brain state was induced at a certain time. For example, if a subject has difficulty falling asleep or experienced restless sleep, and the ambient sensors detect that there was ambient noise and ambient light above a certain threshold, or that the room temperature was too hot or cold, the ambient sensor data obtained using the ambient sensors can provide feedback to the subject or the processing module (described in greater detail below). In some cases, the feedback may comprise a notification to the subject to let the subject know that he or she was restless last night, and that such restlessness may be due to too much ambient light or too much noise in the room at a certain time. In some cases, the feedback may further comprise one or more suggestions to the subject (e.g., a suggestion for the subject to try using an eye mask).

In some cases, the ambient sensor data can be used to determine one or more ambient conditions of an environment, and to adjust one or more environmental conditions (e.g., brightness of light, a noise level of the room, a temperature of room, etc.) based on the one or more detected ambient conditions. In some cases, the one or more environmental conditions can be wirelessly controlled using one or more output devices (e.g., smart lights, smart speakers, and/or smart thermostats). In some cases, the systems of the present disclosure can be configured to communicate with and adjust an operation of the output devices in real-time based on the desired state of the subject at that time or a future time. The desired state can be associated with a particular brightness, noise level, or temperature that is specific to a certain subject or surrounding environment. In some instances, the one or more environmental conditions can be optimized based at least in part on the ambient sensor data. For example, if the system determines that it is best to wake the subject up in a particular stage of sleep (or at a specific time of day), the system can be configured to (i) sense current ambient conditions using the one or more ambient sensors and (ii) adjust the ambient conditions by a predetermined amount to (a) wake up the subject or (b) optimize the environmental conditions for the subject when he or she wakes up or begins to wake up (e.g., by gradually increasing the temperature in the room and/or the brightness of the lights in the room).

In some cases, the ambient sensor data (e.g., ambient sound data) can be used for real-time control or adjustment of device outputs. If a desired state of a subject is to be asleep, and an ambient sensor (e.g., a microphone) picks up ambient noise that is disrupting (or could potentially disrupt) the subject's sleep, the systems of the present disclosure can be configured to play sounds to mask the ambient noise, implement noise cancelling techniques to cancel out the ambient sound, or stimulate the subject's brain to boost whatever state of sleep the subject is in to minimize the chance that the subject will wake up in response to the ambient noise.

In some embodiments, the sensing module can comprise a wearable headband that can be positioned on or around a portion of a subject's head. In some cases, the wearable headband can comprise a central processing unit (CPU), one or more EEG electrodes, and a power source (e.g., a battery). In some cases, the CPU, the one or more EEG electrodes, and the power source may be integrated in one housing. In other embodiments, the sensing module can comprise a device that can be placed in a surrounding environment in which the subject is located. In some cases, the device can comprise a tabletop device. The tabletop device can comprise one or more plug-in wired EEG electrodes configured to connect to a subject's head to obtain EEG brain signals. In some cases, the EEG brain signals obtained using the tabletop device can be provided or transmitted to an external device for processing (e.g., an external desktop, laptop, or any other computing device or unit). In some alternative embodiments, the sensing module can comprise a wearable device component and a tabletop device component. The wearable device component can be configured to communicate with the tabletop device component, and the tabletop device component can be configured to communicatee with the wearable device component. In some cases, the wearable device component can be used to obtain a subject's EEG brain signals and to transmit or provide the EEG brain signals to the tabletop device component for additional processing.

### Processing Module

In some embodiments, the system can comprise a processing module. The processing module can comprise a signal processing module and/or an output device optimization module. The signal processing module can be in communication with the output device optimization module. In some cases, the output device optimization module can be configured to adjust an operation of the output devices (or one or more control parameters associated with the output devices) based on information received from the signal processing module. The information can comprise data associated with one or more properties or characteristics of the one or more biological signals, or one or more biomarkers associated with the biological signals.

### Signal Processing Module

In some embodiments, the processing module can comprise a signal processing module. The signal processing module can comprise one or more processors, ASICs, PLCs, or logic circuits. The signal processing module can be configured to process or analyze one or more biological signals of the subject. As used herein, processing and/or analyzing biological signals (e.g., brain waves and signals) can be more than passive observation, and may include, in some cases, actively monitoring or tracking biological signals or biomarkers associated with such signals. For example, the systems and modules described herein can be used to actively probe for biomarkers to identify abnormal responses to stimulation, including dynamic sensory stimulation. Such abnormal responses may be exhibited in individuals or subjects with one or more neurological or behavioral conditions (e.g. schizophrenia or ADHD) that can cause such individuals or subjects to exhibit different brain responses to sensory or electrical stimulation compared to an average population of individuals (i.e., those who do not have a neurological or behavioral condition) exposed to a similar sensory or electrical stimulation.

### Biological Signals

The signal processing module can be configured to monitor, analyze, process, and/or modulate one or more biological signals to enable neuromodulation and/or neurofeedback. The one or more biological signals can comprise, for example, brain waves or brain signals. The one or more biological signals may comprise one or more signals obtained using any of the sensors or sensing modules described elsewhere herein. The one or more biological signals can comprise an electrical signal and/or an oscillatory signal. The one or more biological signals can be represented as one or more EEG waves or waveforms (also referred to herein as brain waves or brain signals). The one or more biological signals can include an electroencephalogram (EEG) signal, an electromyogram (EMG) signal, an electrocorticogram (ECoG) signal, field potentials within a motor cortex or other regions of the brain, or combinations thereof. The one or more biological signals can correspond to a particular mental state of the subject. For example, in a first mental state, the subject may exhibit a first set of biological signals with a first set of characteristics, whereas in a second mental state, the subject may exhibit a second set of biological signals with a second set of characteristics. The characteristics associated with the biological signals may comprise, for example, a wavelength, a frequency, an amplitude, a phase, a center frequency, a phase difference, a variance, a co-variance, or any other physical property associated with the one or more biological signals.

### Computing Biomarkers

The signal processing module may be configured to process the biological signals to identify one or more properties or characteristics associated with the biological signals (or a subset thereof). The signal processing module may be further configured to compute one or more biomarkers using the characteristics identified for the one or more biological signals.

### Biomarkers

In some embodiments, the signal processing module can be used to compute one or more biomarkers to implement closed-loop, state-based control of device outputs and user stimulation. The biomarkers can correspond to one or more properties or characteristics of a biological signal (or one or more qualitative or quantitative inferences derived from such properties or characteristics) that indicate a particular state or condition of the subject. In some cases, the biomarkers can comprise a series of electrical oscillations that appear within one or more discrete frequency bands for the one or more biological signals obtained using the sensing module.

### Center Frequency

In some embodiments, the biomarkers can comprise a target brainwave oscillation frequency band around a subject's center frequency. The center frequency may correspond to a central frequency between one or more upper and lower cutoff frequencies. In some cases, the upper cutoff frequencies may correspond to a maximum value of one or more biological signals or a spectrum of frequencies associated with the one or more biological signals. In some cases, the lower cutoff frequencies may correspond to a minimum value of one or more biological signals or a spectrum of frequencies associated with the one or more biological signals. In some embodiments, the center frequency can be either an arithmetic mean or a geometric mean of a lower cutoff frequency and an upper cutoff frequency. In some embodiments, the center frequency can be associated with an alpha wave, a beta wave, a gamma wave, a delta wave, a sigma wave, a theta wave, or any other endogenous wave or signal associated with the subject's brain activity.

The signal processing module can be configured to compute the center frequency associated with one or more biological signals of the subject. The center frequency can be computed instantaneously, or with a calibration measure. In cases where the center frequency is computed instantaneously, the center frequency can be determined by applying a transform to the one or more biological signals. In some non-limiting examples, the transform can include, for example, a fast Fourier transform and/or a Hilbert transform. In cases where the center frequency is computed with a calibration measure, the individualized “center frequency” can be computed offline from a 1/f detrended absolute power spectrum corresponding to the subject's brain waves by locating a peak or maximum power within a targeted frequency range. As used herein, a power spectrum may refer to a distribution of power into discrete frequency components that represent or approximate a wave or signal. The power spectrum can be used to determine a statistical average of a wave or signal as analyzed in terms of its frequency content. The 1/f detrended absolute power spectrum may comprise a power spectrum with a corresponding power spectral density (i.e., a power per frequency interval) that is inversely proportional to one or more frequencies of the one or more biological signals.

In some cases, the processing module can be configured to identify a subject's individualized brainwave center frequency within a target range, and to use the individualized brainwave center frequency for closed-loop device control. The individualized center frequency can be computed from the 1/f detrended absolute power spectrum by locating a peak or maximum power within the targeted frequency range. For those individuals that lack a peak or maximum within the targeted frequency range, a center of mass or a midpoint can be used. As used herein, the center of mass may refer to the center of mass of a frequency or power spectrum. The center of mass can be computed as the weighted mean of the frequencies present in a signal, with their magnitudes as the weights. As used herein, the midpoint may refer to a mean or median value associated with one or more biological signals or one or more frequency or power spectrums associated with the one or more biological signals. The individualized center frequency (or the center of mass or midpoint) can be used directly as a discrete biomarker, or as a ratio biomarker to control closed-loop device output on multiple time scales.

In some alternative embodiments, the biomarkers can comprise one or more ratios between two brainwave oscillation frequency bands that define brain-states (e.g., a theta/alpha ratio, a beta/alpha ratio, an alpha/[slow wave+delta wave+theta wave ratio], etc.). In some cases, the biomarkers can comprise a measurement of a coherence between brainwave oscillations recorded from the same electrode and/or across a plurality of different electrodes. In other cases, the biomarkers can comprise brain-state and frequency defined EEG biomarkers (e.g., individualized sigma occurring in stage 2 sleep). In some cases, the biomarkers can comprise non-neural biomarkers from other sensor signals (e.g., heartbeat sensors, pulse oximeters, etc.).

In some cases, the processing module can be configured to continuously filter and smooth unprocessed brain signal data to precisely identify a subject's characteristic target oscillatory frequencies and any corresponding biomarkers.

In some cases, the processing module can be configured to (i) compute multiple individualized oscillatory frequencies and other brain-state biomarkers and (ii) update such computations continuously on varying time scales in order to track a desired brain-state.

In some cases, the processing module can be configured to (i) iteratively compute additional brain-state biomarkers (such as the ratio between two target frequency bands) in a calibration or baseline period and (ii) use the computed brain-state biomarkers to determine minimum, maximum, and intermediate levels needed to set an operating range for device output. As described in greater detail elsewhere herein, the minimum, maximum, and intermediate levels used to set an operating range for device output may correspond to one or more predetermined or adjustable threshold values that are set based on a value or an attribute of one or more biological signals, one or more biomarkers associated with the biological signals, or any power of frequency spectrums associated with the biological signals.

### Output Device Optimization Module

In some embodiments, the processing module can comprise an output device optimization module. The output device optimization module can comprise one or more processors, ASICs, PLCs, or logic circuits. The output device optimization module can be configured to monitor and adjust an operation of one or more output devices in order to (i) control a subject's environment (including exposure to or consumption of various media or sources of digital entertainment) and/or (ii) modulate a subject's physiological, perceptual, cognitive, and/or behavioral states. The output device optimization module may be in communication with the signal processing module via any wired or wireless communication network or protocol.

### Output Devices

The processing module can be in communication with one or more output devices. In some embodiments, the output devices can comprise one or more devices that provide a sensory stimulation to the subject. The sensory stimulation may comprise, for example, visual stimulation, audio stimulation, and/or physical stimulation. In some instances, the sensory stimulation may comprise auditory, visual, electrical, magnetic, vibrotactile, or haptic stimuli. In some cases, electrical stimulation may be applied via surface electrodes (worn on the skin) or subdural electrodes. In other cases, magnetic stimulation may be applied via surface magnets (worn on or near the skin) or subdural magnets. The electrodes used for electrical stimulation and/or the magnets used for magnetic stimulation may be positioned anywhere on or near the user's head. In some alternative embodiments, the output devices can comprise one or more devices that adjust an amount of sensory stimulation provided to the subject. Alternatively, the output devices can comprise one or more devices that change a physical or chemical condition, property, or nature of the environment (e.g., an amount of light in the environment, an amount of noise in the environment, a temperature of the environment, etc.).

In any of the embodiments described herein, the output devices can be controlled to modulate a physiological, perceptual, cognitive and/or behavioral state of the subject and to induce a desired state. In some cases, the desired state can be predicted or inferred based on historical user behavior (e.g., setting an alarm for a certain time every morning), or an occurrence of certain events (e.g., turning off a computer screen or a television after a certain time of day). In some cases, the desired state can be initially set based on the behaviors of other users (e.g., a population of users in a same or similar geographic area). One or more parameters or characteristics of the desired state can be adjusted or refined based on prior or subsequent actions taken by a user or subject. In some cases, the processing modules described herein can be configured to adjust or modify a desired or induced state based on one or more inputs provided by a user or a subject. The one or more inputs provided by the user or the subject may comprise, for example, an indication that the user or subject wishes to be in a particular state, or that the user or subject wishes to change a characteristic or property of the surrounding environment in which the user or subject is located.

In some embodiments, the stimulation can be phase-locked with a detected instantaneous phase of one or more biological signals. In some cases, the stimulation may comprise pulsed stimulation, where pulses of stimulation each occur at, or shortly before, a peak (point of greatest magnitude in a period) of an endogenous wave (e.g., a theta wave, an alpha wave, a delta wave, or any other type of neural oscillation that originates from a subject's brain). In other cases, the pulses of stimulation can each occur at, or shortly before, a trough (point of lowest magnitude in a period) of an endogenous wave (e.g., theta wave, alpha wave, or delta wave). In some alternative embodiments, the pulses of stimulation can each occur at 90 degrees (or slightly more than 90 degrees) before a peak of an endogenous wave (e.g., theta wave, alpha wave, or delta wave). In other alternative embodiments, the pulses of stimulation can each occur at 90 degrees (or slightly more than 90 degrees) before a trough of an endogenous wave (e.g., a theta wave, an alpha wave, or a delta wave).

In some examples, the output devices can comprise one or more adjustable audio speakers or sources configured to transmit or emit audio signals. In other examples, the output devices can comprise one or more adjustable lights or light sources configured to transmit or emit electromagnetic radiation. Alternatively, the output devices can comprise one or more thermostats, secondary computers, television display monitors, transcranial electrical stimulation devices, or household appliances. In any of the embodiments described herein, the output devices can comprise any device that can establish a communication channel with the output device optimization module and control an environment or a state of the subject.

### Thresholds

In some cases, the processing module can be configured to compute and track biomarker minimum and/or maximum levels used for graded or switch thresholding control for one or more output devices. In some cases, the processing module can be configured to determine proportional levels or thresholds for when stimulation should occur or not occur, and a volume or magnitude of an output of the one or more output devices.

In some cases, if a minimum and/or maximum value of a biomarker exceeds a predetermined threshold value, stimulation may be provided or increased. In other cases, if a minimum and/or maximum value of a biomarker exceeds a predetermined threshold value, stimulation may be reduced or eliminated.

In some cases, if a minimum and/or maximum value of a biomarker does not exceed a predetermined threshold value, stimulation may be provided or increased. In other cases, if a minimum and/or maximum value of a biomarker does not exceed a predetermined threshold value, stimulation may be reduced or eliminated.

In any of the embodiments described herein, the predetermined threshold values can be used to determine (i) whether or not to provide stimulation, (ii) a volume or a magnitude of stimulation, or (iii) an amount by which current levels of stimulation should be changed or modified. In any of the embodiments described herein, the predetermined threshold values used to determine (i) whether or not to provide stimulation, (ii) a volume or a magnitude of stimulation, or (iii) an amount by which current levels of stimulation should be changed or modified, may be adjusted based on newly received information (e.g., newly detected biological signals or newly computed biomarkers that are derived based on an analysis of the newly detected biological signals). For example, the predetermined threshold values may be set based on a first set of biological signals received at a first point in time, and may be updated based on a second set of biological signals received at a second point in time. The second point in time may be after the first point in time. In some cases, the predetermined threshold values may be updated in real time as the second set of biological signals are detected, measured, received, or processed.

### Output Device Control

In some cases, the processing module can be configured to control device output in an analog, gradual, or gradient fashion in proportion to a center frequency or a biomarker level. Alternatively, the processing module can be configured to control device output in a digital or “switch” fashion to turn devices “ON” or “OFF” and/or to switch a device between an “ON” state and an “OFF” state. In cases where the device output is controlled in proportion to a detected biomarker level for a particular subject, the processing module can be configured to iteratively compute the biomarker level over a predefined time window and to use the computed biomarker level to drive proportional change in device output. In cases where the control device output is controlled in a switch fashion for “ON” or “OFF” switch device control, the processing module can be configured to iteratively compute biomarker levels to determine when the instantaneous biomarker level crosses a “threshold level,” which can trigger a signal or transmit a command to the output device to turn “ON” or “OFF.” In any of the embodiments described herein, the processing module can be configured to implement both gradient and switch controls, which can be operated in tandem. In any of the embodiments described herein, biomarkers, decision variables (DVs), thresholds, and/or other output device control parameters can begin at arbitrary initial conditions, and the processing module can be configured to update such biomarkers, decision variables (DVs), thresholds, and/or other output device control parameters based on instantaneous and/or cumulative and repetitive user data acquisition.

In some cases, one or more physiological changes can be hallmarked by instantaneous changes in power or level of an absolute or normalized target oscillatory frequency used as a biomarker. In such cases, the processing module can be configured to detect such instantaneous changes in a property, characteristic, or attribute of a particular biomarker (e.g., a target oscillatory frequency) in real time and to modulate a control parameter of an output device based on the instantaneous changes detected or observed. In other cases, physiologic change can be hallmarked by instantaneous changes in ratios of absolute or normalized oscillatory frequencies, such as an individual's “theta/alpha” ratio. In such cases, the processing module can be configured to detect such instantaneous changes in a relationship between two or more select biomarkers (e.g., a first target oscillatory frequency associated with a first type of brain wave and a second target oscillatory frequency associated with a second type of brain wave) and to modulate a control parameter of an output device based on the instantaneous changes detected or observed. In any of the embodiments described herein, the processing module can be configured to compute one or more biomarkers in real-time and to modify peripheral device output in a switch or gradient fashion based on the one or more computed biomarkers.

### Applications

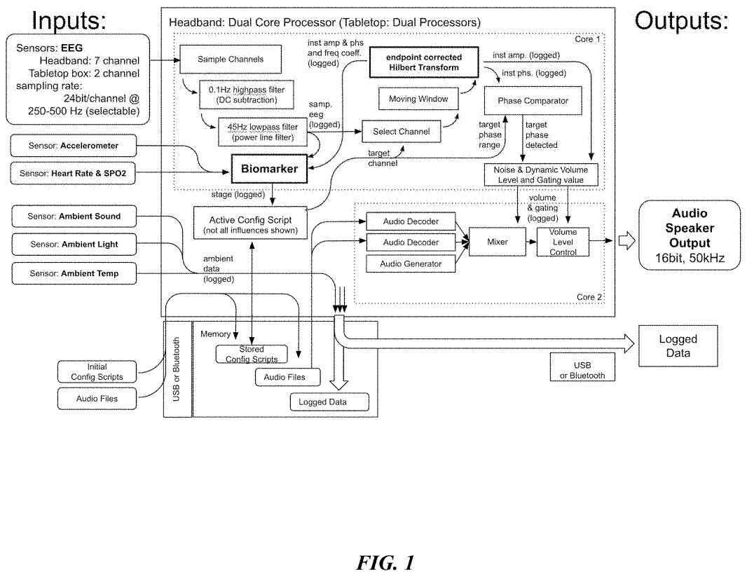

FIG. 1 schematically illustrates a software architecture for recording EEG and computing biomarkers to control audio speaker device output in a real-time closed-loop manner. The software architecture can be implemented for a sensing module (e.g., a wearable headband or a tabletop device) that is in communication with one or more processing modules configured to set and tune control parameters for one or more output devices. The software architecture can be implemented on a multi-channel EEG headband device or a dual-channel research device to instantaneously track EEG brain signals and compute instantaneous biomarkers in order to control audio speaker device output. The left side of the diagram shows a plurality of inputs from an EEG machine, a heart rate monitor, a pulse oximeter, and a plurality of other ambient sensors. Configuration scripts can be used to define initial input and output parameters for brain signal based device control. Active configuration scripts can be set initially based on user preference for output and based on population normative brain signals and corresponding biomarkers. The software architecture can be implemented to iteratively refine configuration parameters for one or more output devices based on instantaneous brain signals and corresponding biomarkers. Such configuration parameters can be refined over the course of a single session, or over the course of a plurality of sessions. The software architecture may be implemented using any of the processing modules or processors described elsewhere herein. In some cases, a first core of a processor can be configured to determine when and how to drive an output of a device (e.g. audio speaker), compute and track the endpoint corrected Hilbert transform (ecHT), and/or compute and track an EEG “biomarker” for a subject. In some cases, a second core of the processor can be configured to generate the audio and drive a low-latency closed-loop playback system. The processing module used to implement the software architecture can be configured to log all the sensor data obtained using a sensing module (as well as many intermediate computational derivations based on the sensor data), which sensor data can be downloaded or live-streamed for additional analysis (e.g., using a computer, a phone, or a cloud based solution).

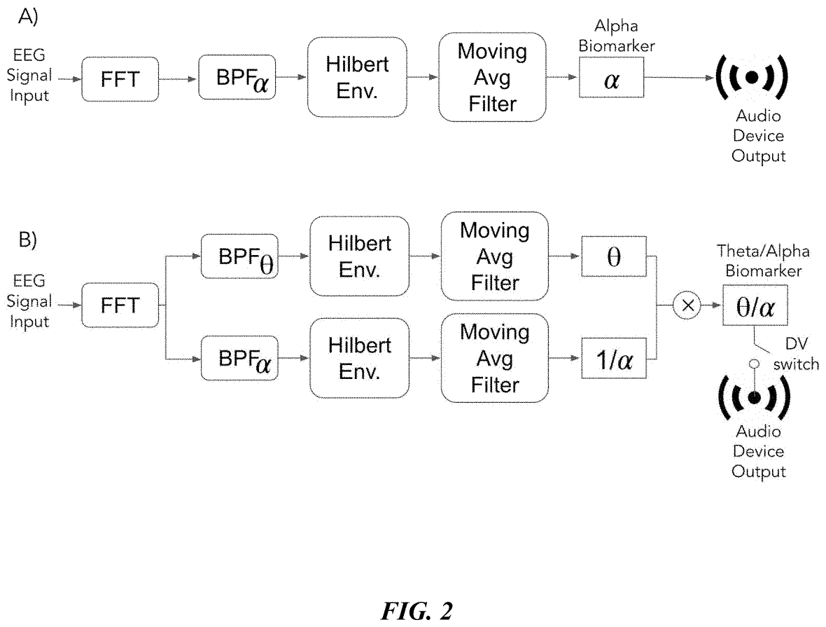

FIG. 2 schematically illustrates a process flow diagram for EEG signal biomarker computation to control audio speaker device output. The process flow diagram represents a logistical flow for the systems signal processing to instantaneously track EEG brain signals and compute in real-time two forms of biomarkers used to control device output (e.g. audio speaker output). In some cases, a sensing module can be configured to measure, detect, or record unprocessed electrical EEG input signals (e.g., voltages) obtained using one or more scalp electrodes. After detection and artifact removal, a processing module can be used to perform a series of operations on the EEG input signals, including but not limited to: (1) a fast Fourier transform (FFT) to decompose the signal into its spectral components; (2) a bandpass filtering (BPF) to select a target spectral range (e.g., the user's own unique alpha spectral band based on prior data or a population alpha band based on population data); (3) an instantaneous endpoint corrected Hilbert transform (ecHT) to recover the Hilbert envelope (Env.) amplitude; and/or (4) a smoothing of the Hilbert envelope using a causal moving average filter that minimizes time delays to generate an instantaneous biomarker that can be used to control device output in a graded and proportional manner. In some cases, the processing module can be configured to control audio speaker device volume in proportion to the biomarker. Devices controlled by the brain signals can be integrated or external to the systems of the present disclosure. For example, audio speaker devices can be mono or stereo and part of a tabletop device or a headband or other wearable device. In some alternative embodiments, similar processes can be performed in parallel on separate spectral bands in order to generate an instantaneous biomarker such as a theta/alpha oscillatory frequency ratio. When the theta/alpha oscillatory frequency ratio crosses an arbitrary, predetermined threshold or a user defined decision variable (DV) level, the processing module can be configured to recognize this event as a trigger or a switch to modulate an audio speaker output or to turn the audio speaker output on or off. The signal processing operations described and illustrated herein can be performed instantaneously and in parallel to yield both continuous graded and step-wise (switch) modulation of devices based on brain biomarkers.

As described above, in some cases an instantaneous endpoint corrected Hilbert transform (ecHT) can be implemented during processing of the one or more biological signals. The ecHT can be used to correct or account for distortions due to Gibbs phenomenon that occur when calculating instantaneous attributes (e.g. an instantaneous phase and/or an instantaneous amplitude) of a signal using a Fast Fourier Transform. The ecHT can be used to determine in real time, based on the sensor readings, the instantaneous phase and instantaneous amplitude of a biological or physiological signal. In some cases, the systems of the present disclosure can correct or account for the Gibbs phenomenon by performing a “frequency domain” ecHT or by performing a “front-padded time domain” ecHT. Both of these approaches can be used to correct the Gibbs phenomenon by ensuring that a signal will be continuous and differentiable at the original end of the signal when a replica of the signal is appended to the signal. In “frequency domain” ecHT, the system can perform a discrete Fourier transform (DFT) to calculate a frequency domain representation of a signal. The system can then apply a causal filter to the frequency domain representation, prior to an inverse discrete Fourier transform (IDFT) step. In “front-padded time domain” ecHT, the system can front-pad the signal with a copy of an end segment of the signal, then apply a causal filter to the padded signal, and then remove the added segment in the time domain, prior to the DFT and IDFT steps. In both of these approaches, the correction is made before the IDFT step that results in an analytic signal. The system can selectively deform the beginning of the signal either in the frequency domain (in “frequency domain” ecHT) or in the time domain (in “front-padded time domain” ecHT) and may not or need not deform the end of the signal. In both “frequency domain” ecHT and “front-padded time domain” ecHT, the value of the end of the signal is not changed, but the value of the beginning of the signal is changed, such that the value of the signal at the beginning and end of the signal is the same. Thus, in some cases, if a replica of the signal is appended to the signal at the original end of the signal, the appended signal is continuously differentiable at the original end of the signal. By removing the jump discontinuity at that point, the processing module can eliminate (or significantly reduce) the Gibbs phenomenon distortions at the end of the analytic signal that results from taking an IDFT. This can allow the processing module to accurately calculate instantaneous phase and instantaneous amplitude of a signal.

In some cases, the processing module can be configured to correct or adjust for the Gibbs phenomenon by performing an “end-padded time domain” ecHT. In this approach, the processing module can append a segment of data values (e.g., zeros) of at least one period length to the end of a signal and then apply a causal filter, which has a directionality property, to make the padded signal continuous and differentiable at the endpoint of the original signal without deforming the original end of the signal. By pushing away the end of the padded signal from the original end before the DFT procedure, the processing module can ensure that the Gibbs distortion occurs away from the original end of the signal. Again, this can allow the processing module to accurately calculate an instantaneous phase and an instantaneous amplitude of a signal.

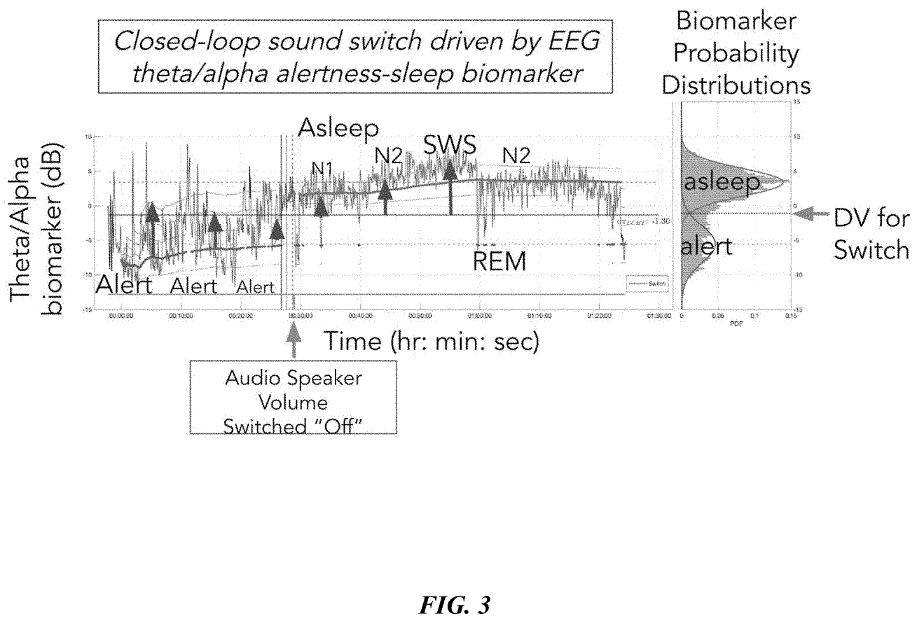

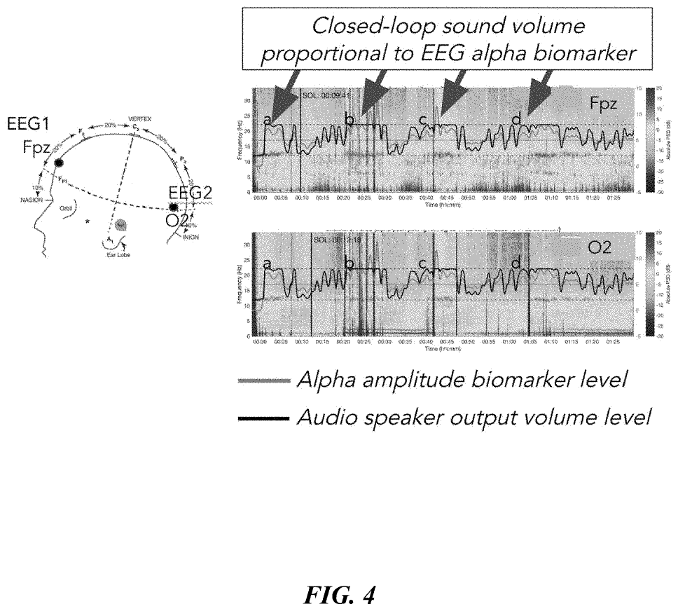

FIG. 3 schematically illustrates a system for instantaneously tracking EEG brain signal from a single electrode in order to compute real-time biomarkers of alertness versus sleepiness brain-states (e.g. theta/alpha ratio). In some cases, cumulative probability distributions of the biomarker can be used to determine an individual subject's probability of being alert versus asleep. In one example, the subject's eyes can be closed and the target biomarker of interest can comprise the mean ratio between theta and alpha oscillation frequency bands. When the biomarker exceeds a predetermined decision variable (DV) level, the system can be configured to activate a switch to turn one or more audio speakers off. These operations can be used in isolation or in combination with additional graded speaker control or with closed-loop phase-locked audio pulse delivery (e.g. as illustrated in FIG. 4). In some embodiments, the mean and standard deviations can be used to track graded instantaneous changes in levels of alertness and sleepiness. As shown in FIG. 3, progressive sleep states for stage 1, stage 2, Slow Wave Sleep and Rapid-Eye-Movement can be indicated with labels: N1, N2, SWS and REM, respectively. The cumulative “Probability Distributions” and resulting DV based on the user's EEG output can be computed iteratively with a graded descent operation during continued data acquisition. In some embodiments, when a target biomarker crosses an arbitrary or individualized DV threshold level indicating transition from an alert state to a sleep state, the audio headphone speakers can be switched to an “Off” state within a predetermined time period (e.g., 60 second after detecting the transition). The switching off of the audible speaker output can be confirmed by an experimental observer as well as by subjective reports from the subject that they were “dozing off” when the sound was turned off. If the EEG biomarker indicates a return to an alert state, the speaker switch operation can be reversed to automatically turn “On” the audio speakers again.

FIG. 4 schematically illustrates a system for instantaneously tracking EEG brain biomarkers at separate Frontal (Fpz) and Occipital (O2) channel locations in order to control audible changes in speaker output volume with volitional change in focused alertness (i.e., with closed-loop audible neurofeedback). In one example, the subject's eyes can be closed and the target biomarker of alertness, hyperarousal, or vigilance can comprise the mean alpha oscillation frequency band amplitude that is extracted from the absolute power spectral density (PSD) for the corresponding band delimited alpha frequency range. The audio speaker volume output can be varied in proportion to a user's individualized maximum and minimum alpha signal levels. The user's maximum and minimum mean alpha biomarker levels can be set to values of, for example, 8 dB and 3 dB, respectively, as determined in a prior data calibration session. The maximum and minimum alpha biomarker levels can indicate maximal and minimal alertness levels, respectively. The user can practice alternately increasing and decreasing the audio speaker volume on 4 separate occasions (a, b, c and d) within a 1.5 hour session. To do so, the user simply focuses their attention on their forehead to increase alpha and as their attention relaxes audible feedback indicates relaxation. Here, the Frontal EEG electrode signal (EEG1) is used to control audio speaker device volume. However, Occipital EEG or some combination as well as coherences across EEG electrode signals can be used to set the biomarker levels that drive audio speaker output. Artifacts may or may not be removed or rejected prior to computing the biomarker level. If artifacts are not removed or rejected, there may be large amplitude artifact signals that cross the entire frequency range of the frequency-time (spectrogram) plot. In some instances, the alpha biomarker level can artifactually supersede its natural maximum level, which indicates that the processing methods disclosed herein can be implemented successfully regardless of movement artifacts. In other instances, the systems of the present disclosure can be configured to remove artifacts in situ to further optimize fine grain control of the audio speaker output.

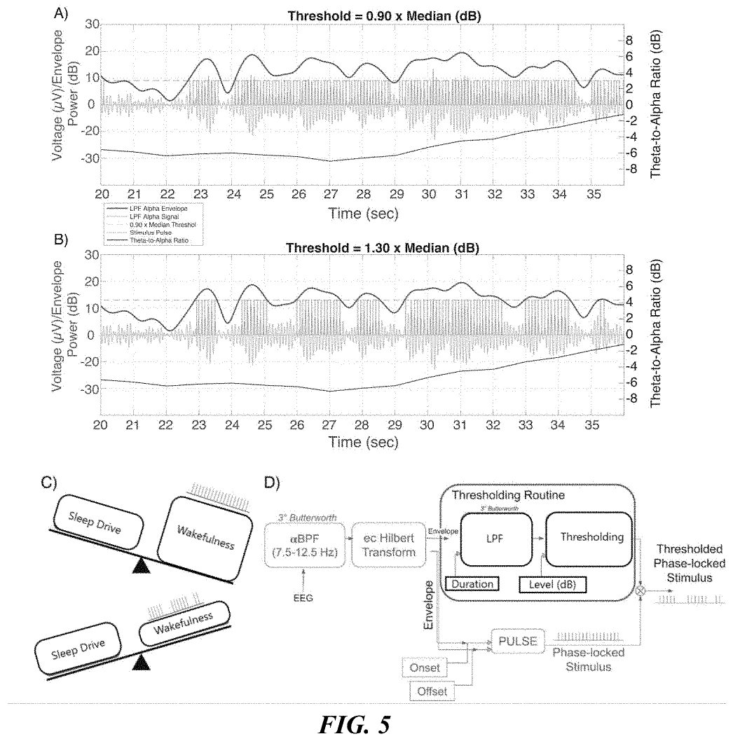

FIG. 5 schematically illustrates a system for instantaneously tracking EEG signals and computing a low frequency (LF) normalized alpha envelope biomarker to set a threshold level for delivery of closed-loop audio output phase-locked to the peak phase of alpha brainwave oscillations. As shown in (A), a low biomarker threshold level (0.9) can allow for high rate closed-loop audio pulse output from the audio speaker. As shown in (B), a high biomarker threshold level (1.3) can allow for a lower rate of closed-loop audio pulse output restricted to times when the alpha signal is closer to maximal amplitude, which can indicate a highly alert brain-state with eyes-closed. As shown in (C), changes in the magnitude of the EEG biomarker for wakefulness can be used to track and modulate the balance between sleep drive and wakefulness with audible speaker output. As shown in (D), a combination of conditional operations can be used to deliver phase-locked close-loop audio conditional on the instantaneous alpha biomarker.

In another aspect, the present disclosure provides a system for modulating brain states. The system may comprise: (a) a sensing module comprising one or more sensors configured to detect one or more biological signals of a subject, wherein at least one of the one or more sensors is placed in contact with a portion of the subject's body; (b) a signal processing module in communication with the sensing module, wherein the signal processing module is configured to compute one or more biomarkers based on the one or more biological signals; and (c) an output device optimization module in communication with the signal processing module and one or more output devices configured to provide a stimulation to the subject. The output device optimization module can be configured to: (i) determine an optimal stimulation to induce a predetermined desired state in the subject, based on the one or more biomarkers and one or more reference biomarkers associated with the predetermined desired state, (ii) control an operation of the one or more output devices to provide the optimal stimulation to the subject, (iii) iteratively update the optimal stimulation based on a detection of one or more instantaneous changes to the one or more biomarkers, and (iv) modify the operation of the one or more output devices in real time to provide the updated optimal stimulation to the subject to induce the desired state in the subject.

In some embodiments, the predetermined desired state can be predicted or inferred based on historical user behavior (e.g., setting an alarm for a certain time every morning), or an occurrence of certain events (e.g., turning off a computer screen or a television after a certain time of day). In some cases, the predetermined desired state can be initially set by the subject (e.g., based on one or more inputs or preferences provided or articulated by the user). In other cases, the predetermined desired state can be initially set based on the behaviors of other users (e.g., a population of users in a same or similar geographic area).

The predetermined desired state can be associated with one or more reference biomarkers. The presence or detection of such reference biomarkers can indicate that a subject is in the desired state. The absence or lack of detection of such reference biomarkers can indicate that the subject is not in the predetermined desired state.

The output device optimization module can be configured to determine an optimal stimulation to induce the predetermined desired state in the subject. The optimal stimulation can be determined based on a difference or a comparison between the one or more computed biomarkers and the one or more reference biomarkers associated with the predetermined desired state. The optimal stimulation can be iteratively updated such that the subject eventually exhibits one or more biomarkers that are the same as or similar to the one or more reference biomarkers after exposure to the optimal stimulation and/or the updated optimal stimulation. The optimal stimulation can be iteratively updated such that a difference between the one or more biomarkers exhibited by the subject and the one or more reference biomarkers is gradually reduced or minimized.

In another aspect, the present disclosure provides a method for modulating brain states. The method can comprise: a) using one or more sensors to detect one or more biological signals of a subject; (b) computing one or more biomarkers based on the one or more biological signals; (c) determining an optimal stimulation to induce a predetermined desired state in the subject, based on (i) the one or more biomarkers and (ii) a set of reference biomarkers associated with the desired state; (d) controlling an operation of one or more output devices to provide the optimal stimulation to the subject; (e) iteratively updating the optimal stimulation based on a detection of one or more instantaneous changes to the one or more biomarkers; and (f) modifying the operation of the one or more output devices in real time to provide the updated optimal stimulation to the subject to induce the desired state in the subject.

### Bio-Controlled Devices and Environments

In another aspect, the present disclosure provides bio-controlled and/or bio-influenced systems. The bio-controlled and/or bio-influenced systems of the present disclosure may comprise one or more closed-loop systems.

In some instances, based on either the time of day or the desired state that a subject wants to be in (asleep, awake, relaxed, focused, etc), sensor data (e.g., brainwaves, brain state, body temperature, heart rate, etc.) may be collected from a subject's body using any of the sensing modules described herein. The sensor data may be processed and used to alter a subject's physical environment to the optimal conditions needed to effect a desired state. The sensor data may be processed using any of the processing modules described elsewhere herein.

In one example, a subject can be in bed and may be asleep or trying to fall asleep. The sensing module can read a body temperature of the subject and determine that the subject's body temperature is too hot for optimal sleep conditions. The sensing module can communicate with a thermostat via a wireless or wired communication network to reduce room temperature. In some cases, the sensing module can communicate with a temperature controllable mattress or pillow, to reduce a temperature of the mattress or pillow.

In another example, a subject's desired state can be to relax, wind down, and/or prepare for sleep. In such cases, the sensing module can be used to determine a room brightness. If the sensing module determines that the room brightness is too bright, or that there is too much blue light, the sensing module can communicate with one or more controllable light sources to dim the light sources or to modify the lighting emitted by the light sources (e.g., by adjusting a color or a temperature of the light). In some cases, the sensing module can be used to read or monitor a subject's EEG to determine that the subject is in an awakened state. In such cases, the sensing module can be configured to (i) directly stimulate peak alpha or (ii) transmit a command to an external device to stimulate peak alpha, in order to induce a transition from a wakefulness state to a sleep state.

In another example, a subject's desired state can be to wake up feeling refreshed. In such cases, the sensing module can be used to read or monitor a subject's EEG to determine that the subject is in a sleep state. In some cases, the sensing module can be configured to (i) directly stimulate trough alpha or (ii) transmit a command to an external device to stimulate trough alpha, in order to induce a transition from a sleep state to a wakefulness state at an optimal time within the subject's sleep cycle. In some cases, the sensing module or a processing module in communication with the sensing module can be configured to signal to an application or one or more controllable room lights to increase a room brightness. In some cases, the sensing module or a processing module in communication with the sensing module can be configured to signal to one or more controllable window shades in a subject's room to rise and/or fall. In some cases, the sensing module or a processing module in communication with the sensing module can be configured to signal to a coffee maker to prepare coffee. In some cases, the sensing module or a processing module in communication with the sensing module can be configured to signal to a shower to turn on. In any of the embodiments herein, the sensing module or the processing module in communication with the sensing module can be configured to control an operation of one or more shades, a thermostat, lighting, speakers, a coffee maker, a smoothie maker, a shower, a television, a radio, car lighting, car audio devices, and/or any other device that the subject can use to control his or her environment. In some cases, the controlling the operation of one or more devices can comprise turning the device on and/or off, turning a volume up and/or down, toggling a do not disturb mode to turn notifications on or off, or adjusting lighting and music level based on a level of alertness of the subject. In some cases, the sensing module or the processing module in communication with the sensing module can be configured to provide push notifications to the subject (e.g., through the subject's phone or through an application installed on the subject's phone) to recommend actions based on a time of day. The recommended actions may include, for example, working out at a certain time based on the subject's goal bedtime and wake time, meditating at a certain time based on the subject's goals, or eating at a certain time based on one or more brainwaves detected for the subject.

### Computer Systems

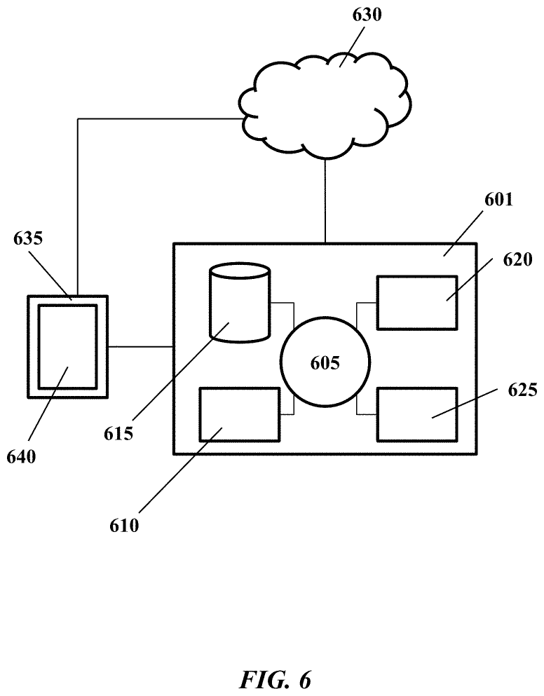

In an aspect, the present disclosure provides computer systems that are programmed or otherwise configured to implement a method for optimizing device outputs based on one or more biological signals to achieve a desired brain state. FIG. 6 shows a computer system 601 that is programmed or otherwise configured to implement such a method for optimizing device outputs. The computer system 601 may be configured to, for example, receive one or more biological signals using a sensing module, process the one or more biological signals using a processing module to compute one or more biomarkers, and to use at least the one or more computed biomarkers to control an operation of one or more output devices. The computer system 601 can be an electronic device of a user or a computer system that is remotely located with respect to the electronic device. The electronic device can be a mobile electronic device.

The computer system 601 may include a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are located external to the computer system 601 (e.g., on a remote server that is in communication with the computer system 601 through an intranet or the Internet).

The computer system 601 can communicate with one or more remote computer systems through the network 630. For instance, the computer system 601 can communicate with a remote computer system of a user (e.g., a human subject). Examples of remote computer systems include personal computers (e.g., portable PC), slate or tablet PC's (e.g., Apple® iPad, Samsung® Galati Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a portal for a subject to monitor or track one or more biological signals obtained using any of the sensing modules described herein, or to control an operation of one or more output devices). The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. For example, the algorithm may be configured to process one or more biological signals measured or detected using a sensing module to compute one or more biomarkers, and to use at least the one or more computed biomarkers to control an operation of any one or more output devices described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A system for controlling one or more output devices, comprising:
(a) a sensing module comprising (i) one or more sensors to detect at least one of a biological parameter of a subject and a biological signal of the subject upon contact with a portion of the subject's body, and (ii) an additional sensor to detect one or more ambient conditions associated with a surrounding environment of the subject;
(b) a signal processing module in communication with the sensing module, wherein the signal processing module is configured to aggregate and process data obtained using the one or more sensors to compute one or more markers for the subject; and
(c) an output device optimization module in communication with the signal processing module and the one or more output devices, wherein the output device optimization module is configured to determine an optimal output for the one or more output devices and control an operation of the one or more output devices to provide the optimal output based on (i) the one or more computed markers and (ii) data obtained using the additional sensor.

2. The system of claim 1, wherein the one or more sensors comprise a sensor to detect the biological signal of the subject, wherein the sensor comprises an electrode, a surgically implanted electrode, a surface electrode, or an encephalogram (EEG) electrode.

3. The system of claim 1, wherein the biological signal comprises an electroencephalogram (EEG) signal, an electromyogram (EMG) signal, an electrocorticogram (ECoG) signal, or a field potential within a cerebral cortex region of the subject's brain.

4. The system of claim 1, wherein the one or more sensors comprise a sensor to detect the biological parameter of the subject.

5. The system of claim 1, wherein the additional sensor comprises a sensor configured to detect one or more environmental conditions of the surrounding environment.

6. The system of claim 1, wherein the biological parameter comprises a physical or physiological condition, state, or property of the subject.

7. The system of claim 1, wherein the one or more ambient conditions correspond to a temperature of the surrounding environment, an amount or volume of sound or noise in the surrounding environment, a humidity of the surrounding environment, an air quality in the surrounding environment, or a lighting condition of the surrounding environment, wherein the lighting condition comprises an amount, an intensity, a directionality, a color, or a temperature of light in the surrounding environment.

8. The system of claim 1, wherein the one or more markers comprise a center frequency of the biological signal.

9. The system of claim 8, wherein the center frequency is computed by applying a transform to the biological signal.

10. The system of claim 8, wherein the center frequency is computed using a 1/f detrended absolute power spectrum by locating a peak or maximum power within a targeted frequency range of the biological signal.

11. The system of claim 1, wherein the one or more markers comprise a ratio between two or more brainwave oscillation frequency bands.

12. The system of claim 1, wherein the one or more markers comprise a measurement of a coherence between two or more brainwave oscillations.

13. The system of claim 1, wherein the one or more markers comprise a measurement of a phase shift or a phase difference between two or more brainwave oscillations.

14. The system of claim 1, wherein the one or more markers comprise a variance or a covariance associated with one or more brainwave oscillations.

15. The system of claim 1, wherein the output device optimization module is configured to operate or control the one or more output devices in a graded proportional manner.

16. The system of claim 1, wherein the output device optimization module is configured to operate or control the one or more output devices in a switch fashion.

17. The system of claim 1, wherein the output device optimization module is configured to implement an optimization framework for active suppression or amplification of neural oscillations over one or more time-scales using closed-loop stimulation.

18. The system of claim 1, wherein the output device optimization module is configured to control the one or more output devices based on one or more threshold values associated with the one or more markers.

19. A method for controlling one or more output devices, comprising:
(a) using (i) one or more sensors to detect at least one of a biological parameter of a subject and a biological signal of the subject and (ii) an additional sensor to detect one or more ambient conditions associated with a surrounding environment of the subject, wherein at least one of the one or more sensors is placed in contact with a portion of the subject's body;
(b) processing the data obtained using the one or more sensors to compute one or more markers for the subject; and
(c) controlling an operation of the one or more output devices based on the one or more computed markers and data obtained using the additional sensor.

20. A system for modulating brain states, comprising:
(a) a sensing module comprising (i) one or more sensors to detect at least one of a biological parameter of a subject and a biological signal of the subject upon contact with a portion of the subject's body, and (ii) an additional sensor to detect one or more ambient conditions associated with a surrounding environment of the subject, wherein at least one sensor of the sensing module is configured to contact a portion of the subject's body;
(b) a signal processing module in communication with the sensing module, wherein the signal processing module is configured to aggregate and process data obtained using the one or more sensors to compute one or more markers for the subject; and
(c) an output device optimization module in communication with the signal processing module and one or more output devices, wherein the output device optimization module is configured to determine an optimal output for the one or more output devices and control an operation of the one or more output devices to provide the optimal output based on (i) the one or more computed markers and (ii) data obtained using the additional sensor, wherein the one or more output devices are configured to provide the subject with a stimulation to change a current state of the subject or to induce a desired state in the subject.
